(19) United States
(12) Patent Application Publication
(10) Pub. No.: US 2016/0346940 A1
(43) Pub. Date: Dec. 1, 2016

(54) MODULAR BASE LINK FOR A COUNTERBALANCING ARM

(71) Applicant: CENTRE FOR IMAGING TECHNOLOGY COMMERCIALIZATION (CIMTEC), London (CA)

(72) Inventors: Jeffrey Scott BAX, London (CA); Christopher Stanley Robert WARING, London (CA); Aaron FENSTER, London (CA)

(21) Appl. No.: 15/117,255

(22) PCT Filed: Feb. 7, 2014
(86) PCT No.: PCT/CA2014/000101

§ 371 Date: Aug. 8, 2016

(51) Int. Cl. B25J 19/00; A61B 90/50; A61B 34/30; A61B 90/11

(52) U.S. Cl. CPC B25J 19/0016; A61B 90/11; A61B 90/50; A61B 34/30; F16M 2200/041; F16M 2200/044; F16M 2200/063; A61B 2090/374

(57) ABSTRACT

Disclosed is a modular base link for use in a counterbalancing arm. The base link includes two base plates and four connection points. Two stabilizing members are connected to and extend between the base plates. Two resilient members, one of which is hingeably connected to and in communication with the two connection points, the other resilient member is hingeably connected to and in communication with the other connection points. The connection points are eccentrically and orthogonally disposed relative to each other. The resilient members are sufficiently resilient to permit movement of the base plates relative to each other so as to counterbalance the arm when a load is applied to either base plate.

### TECHNICAL FIELD

The present relates to counterbalancing arms, and more particularly to a modular base link for use with counterbalancing arms.

### BACKGROUND

In image-guided surgical interventions, an apparatus that aids a physician in precisely aligning a needle with an intended target; especially within confined spaces of the operative field, is needed. A problem that is specific to the medical field relates to magnetic resonance image (MRI)-guided devices, many of which have been developed to perform needle guidance in the delivery of cancer treatment and biopsies within the prostate. Typically, these devices are too large to operate within the confined space of the MR scanner bore and require removal of the patient from the bore to perform interventions. Removal of the patient from the bore interrupts the workflow increasing the time to complete procedures and provides an opportunity for targets to shift thereby reducing accuracy. As a result, interest has developed in compact devices capable of completing interventions within the scanner bore.

Also, work-related musculoskeletal disorders are a widespread problem amongst diagnostic medical technologists. Furthermore, within certain industrial settings poor worker ergonomics adversely affects productivity and health and safety. Heavy tools or parts may require maneuvering in repetitive or awkward motions. Workers may also be required to maintain fixed poses for extended periods of time. To improve worker ergonomics, devices have been developed to support and position payloads, including tools or parts; specifically to counterbalance the payloads. These devices counteract the force of gravity to simulate the tool floating in air and improve worker ergonomics.

Equipoised or articulating arm structures attached either to the user via harness or a support fixture (i.e. wall, pillar), which use a combination of a parallelogram linkage mechanism and a spring counterbalance system to counterbalance a payload or weight, are known in the art. Articulating arm types are used in various industries including medical, dental, optic, manufacturing for heavy lifting and repetitive tasks, machine tooling and robotic applications. A number of designs of such arm types are known and are described below.

U.S. Pat. No. 5,435,515 for ADJUSTABLE, ISO-ELASTIC SUPPORT APPARATUS to Diguilio et al., disclose an adjustable, isoelastic support arm for a stabilizing device which can operate in conjunction with camera equipment to obtain stabilized motion picture film or video images. The arm consists a section which included a series of pivotally interconnected links defining a parallelogram; adjustable tensioning means for providing forces for weight support; and means for adjusting the tension means to provide weight support forces which are contoured to articulation of the weight support means for lateral and vertical movements of the weight support means. This particular tensioning assembly permits continuous adjustment of the geometric relationship between the endpoints of the tensioning assembly and the remaining structures which comprise the support arm. This can include adjustment of the frame of the support arm, or adjustment of an end point of the tensioning assembly relative to the frame of the support arm using a cable and drum arrangement coupled with a spring of appropriate size and tension.

The tensioning means includes one spring attached to and extending along a second link of the series of interconnecting links which is adjacent to and pivotally connected to the first link, one pulley for receiving a cable extending from and between the two springs extending along the second ling, a second pulley for receiving a cable extending form and between the second spring and a third spring extending form the second drum to the attachment point. Generally, the lifting capability of the support arm is adjusted for lighter loads by shortening the height of the effective parallelogram defined by a given arm section (upper arm and/or forearm), and vice versa (for heavier loads). This causes a reduction in spring tension, and in the differential in spring stretch throughout the excursion of the defined parallelogram, which increases the effective iso-elasticity of the support arm.

U.S. Pat. No. 7,618,016 for EQUIPOISING SUPPORT APPARATUS to Brown, G. W., discloses a parallelogram equipoising support arm for camera stabilizing devices that is adjustable. The support arm consists of springs that do not have an appropriate rate (offset variably outside, as well as inside, the lifting triangle) and can actively provide for varying the contour of isoelasticity established for the support arm, substantially independently of the adjustment for supporting cameras of different weights. The support arm also consists of a lifting triangle operating in conjunction with a parallelogram support arm and comprising a substantially vertical shorter side, a longer side and another side that consists of a flexible resilient member, the expansion or contraction of which pivotally biases the apex angle of the sides (and thus the associated parallelogram) from its most obtuse form, up past the condition of being a right angle and on up to its most acute form. The spring offset can be actively adjusted relative to the parallelogram position so that the lift is selectably appropriate throughout the range.

U.S. Pat. No. 7,543,518 for LINK ASSEMBLY FOR A SNAKE LIKE ROBOT ARM to Buckingham et al., relates to a link assembly for a robot arm or snake arm which consists of a series of link members/segment (two or more) joined end to end to produce an arm of the appropriate length for the intended purpose. This “snake-like” arm has the ability to be manipulated to flow axially along its length and to follow a convoluted path in the manner of a snake. Each of the links is adapted for limited movement with respect to the other and resilient elastomeric material disposed between them and bonded or keyed to them whereby movement between the two link members results in shear movement within the elastomeric material disposed between them. A control wire is used for controlling the movement of multiple links within the segment wherein the control wire controls the operation of each segment. It maintains the links under tension or compression. At least one of the members of each link may be provided with means for guiding the wires from one end of the segment to the other. The wires may be disposed externally of the segment links. Each wire may terminate in a ferrule, which is adapted to engage with a corresponding recess in the end cap of a segment so that on tensioning the wires, the ferrule is brought into engagement with the end cap to exert a compressive load an each of the segments to maintain the stiffness of the links in the segment. Each of the three control wires may be operated/controlled by an actuator: where there are control wires for a plurality of segments. Depending on the variants in the tension the individual links will seek to move in response to the changing tension in the wires thereby producing movement in the segments to permit guidance of the segment end to a given location in the work place.

U.S. Pat. No. 7,837,674 for COMPACT COUNTER BALANCE FOR ROBOTIC SURGICAL SYSTEMS to Cooper, T. G., teaches a counterbalanced set-up arm to support a robotic arm (and multiple joint arms), including a linkage and a spring-cable-pulley balancing mechanism coupled to the linkage around a pivotal joint. The linkage couples to a support structure at one end and supports a weight at the other end. The counterbalance mechanism consists of two parallel links connected between brackets at the support and payload ends. The two links don't share a common pivot and each have one pivot on each bracket for a total of four pivots. The cable is further coupled to the end of a compression spring to form a tension in the cable to counter balance a weight applied at an end of the second link. The additional weight of additional links and an attached robotic surgical arm may also be balanced out by a counter balancing force with an appropriate choice of spring constant K and cabling that is capable of withstanding the additional forces applied. As the linkage is deformed to vertically adjust the height of the weight with a different moment arm length, the spring-cable-pulley balancing mechanism varies a cable path length to modify the compression of a spring and a tension in a cable to adjust the amount of counter balance force applied to the linkage.

U.S. Pat. No. 8,066,251 for EQUIPOISING SUPPORT APPARATUS to Brown, G. W., discloses a parallelogram equipoising support arm for camera stabilizing devices, including a pair of parallel upper arm and forearm links and a tensioning assembly that can provide two different fixed adjustments and one automatic adjustment to the geometric relationship between the end point of the tensioning assembly and the remaining structures that comprise the support arm. This structure provides a consistent lifting force by means of a resilient member of appropriate dimension but not necessarily appropriate “spring rate”. In each parallelogram segment, a spring is used to counterbalance the payload and any subsequent segments in the arm. contained in U.S. Pat. No. 8,066,251. The use of pulleys and multiple springs in each arm is the prior art which this patent is attempting to improve/replace. This design presented in this patent uses a single spring and no pulleys to counterbalance the payload. The forearm link has a similar configuration to that of the upper arm link. The preload of the spring in each segment can be adjusted to accommodate payloads of varying weights. As the payload travels away from the horizontal to extreme angles, a “rate” adjustment is provided to the spring counterbalance. The rate adjustment causes the attachment point of the spring to swing inwards and outwards as the arm is moved up and down. The swinging of the attachment point changes of output force of the spring and offers some correction to counterbalancing errors. It can operate at angles of ±70 degrees with the use of this correction. It requires the adjustment of two setting, lift and rate, for each segment in the arm. Each time the arm payload is changed, the two adjustments on each segment must also be changed.

Published US patent application no. US 2005/0193451 for ARTICULATING ARM FOR MEDICAL PROCEDURES to Quistgaard et al., disclose an apparatus for precise positioning of a medical device (i.e. therapy head) or use of a therapy head over a patient body for an extended period of time. The apparatus comprises an articulating arm, a positional encoder incorporated into the arm and a means for load balancing. The apparatus may also include a robotic driver and an additional rhythmic motion sensor. The arm comprises two or more segments, and a load balancing mechanism is used between each segment either independently (each segment is self-balancing with respect to the other segments of the arm) or dependently (each segment balances in combination with one or more adjacent segments). Load balancing for the distal most arm segment must also adjust for the therapy head and any positional changes it may create during a medical procedure. The range of motion of the arm itself is restricted to prevent the arm from becoming unbalanced. The load balancing mechanism includes either one or more cooperative motors or multiple springs and counterbalancing weights. The load balancing mechanism compensates for both the load of the therapy head and the change in the center of gravity as the therapy head is extended away from the base in a horizontal plane (the most unbalancing configuration). The load balancing mechanism also compensates for any hysteresis that may accompany the movement of the arm. Thus the greater the ability of the load balancing means the greater range of motion allowable on the articulating arm. A force generating device is provided to provide sufficient resistance force to the arm to hold the arm in position after it is moved into place.

There are many known articulating arms that are configured to support a device of varying masses, but most have significant drawbacks. Some of these known arms use a coiled spring having a fixed spring rate as described in U.S. Pat. No. 8,066,251. for a given payload a spring possessing a specific uniform spring rate is required. In these arms, when the mass is varied, the coiled spring assembly disadvantageously cannot be adjusted. Many of these arms use a spring-cable-pulley system; particularly with arms consisting of a series of interconnecting links as the type described in U.S. Pat. Nos. 5,435,515, 7,618,016, and 7,837,674. It is also known to use torsion springs in joints of the arm to generate torques which counter the load torques in the joints of the arm. Furthermore, the concept of using a combination of springs and weights to counterbalance a payload is known as described in published US Application No. 2005/0193451. A link assembly for a robot arm or snake arm consisting of two or more link members/segments in series that can be manipulated to flow axially along its length to guide a segment end to a given location is known as described in U.S. Pat. No. 7,543,518. Also, a counterbalanced set-up arm to support a robot arm comprised of multiple joint arms, including a linkage and spring-cable-pulley balancing mechanism is known as taught by U.S. Pat. No. 7,837,674.

It is therefore desirable to reduce many of the aggravating factors reported by workers in the above-noted fields.

### BRIEF SUMMARY

We have designed a modular base link for use in a counterbalancing arm (a serial chain manipulator) which significantly reduces, or essentially eliminates, the drawbacks of the designs described above. The base link provides the arm with two degrees of freedom (pitch and yaw) for counterbalancing the weight and positioning of a tool or any payload located on the end of the arm. Tools on the end of the arm can be translated and rotated by the human operator and will remain in position once the operator releases the arm. Furthermore, since our arm counterbalances the weight of the tool, the force a human operator must exert to adjust the tool position is substantially reduced.

Our counterbalance arm can also be used to support payloads (i.e. an imaging probe) for medical applications, specifically image-guided interventions. The arm can be used either passively or robotically. An additional advantage of our design is that it can handle heavy payloads. Various aggravating or work-related disorders come from people handling payloads that are unsupported. Our design supports the payload so that the user does not over-stress or over-extend their reach. The advantage of the passive system is that it substantially reduces or essentially eliminates stress on the user when handling heavy payloads. Alternatively, the system also reduces the stress of holding a light payload for a long period of time. As a robotic device, safety is improved because the device is self-supporting and there is no need for large motors to support the weight of the robot.

In one aspect, there is provided a modular base link for use in a counterbalancing arm, the base link comprising:

a first base plate;

a second base plate;

first and second connection points;

third and fourth connection points;

at least two stabilizing members connected to and extending between the first and second base plates; and at least two resilient members, the first resilient member being hingeably connected to and in communication with the first and second connection points, the second resilient member being hingeably connected to and in communication with the third and fourth connection points, the connection points being eccentrically and orthogonally disposed relative to each other, the resilient members being sufficiently resilient to permit movement of the base plates relative to each other so as to counterbalance the arm when a payload is applied to either base plate.

According to another aspect, there is provided a modular base link for use in a counterbalancing arm, the base link comprising:

a body;

at least one wheel assembly having first and second spaced apart wheels and a first endless loop mounted on the first and second wheels, the first wheel assembly being fixably mounted on a support, the first wheel assembly being in communication with the body;

an eccentric cam assembly connected to the first wheel, the eccentric cam assembly having a plurality of eccentric cams that are orthogonally disposed relative to each other; and

at least two resilient members extending between the body and the eccentric cam assembly, one end of each resilient member being connected to the body, the other end of each resilient member being abuttingly connected to the eccentric cam assembly,

the body being located for orbital rotation about the first wheel and the eccentric cam assembly so as to cause the resilient members to travel along the eccentric cam assembly.

According to another aspect, there is provided a link comprising:

a base;

at least two parallel support arms hingeably connected to the base;

a first parallelogram assembly having at least two substantially parallel arms hingeably connected to the support arms at four arm connection points; and

at least two resilient members located in substantial parallelism, one resilient member being hingeably connected to and in communication with first and second resilient member connection points, the other resilient member being hingeably connected to and in communication with third and fourth resilient member connection points, the connection points being eccentrically and orthogonally disposed relative to each other, the resilient members being sufficiently resilient to permit movement of the first and second support arms relative to each other.

According to another aspect there is provided a counterbalancing arm for payload positioning, the arm comprising:

at least two modular base links connected together for movement relative to each other, each base link having: i) two base plates; ii) four connection points; iii) at least two stabilizing members connected to and extending between the base plates; and iv) at least two resilient members, the first resilient member being hingeably connected to and in communication with two of the connection points, the second resilient member being hingeably connected to and in communication with the other two connection points, the connection points being eccentrically and orthogonally disposed relative to each other, the resilient members being sufficiently resilient to permit movement of the base plates relative to each other so as to counterbalance the arm when a payload is applied to either base plate.

According to another aspect there is proving a counterbalancing arm comprising:

first and second base plates, each base plate having two spaced apart connection points;

a central stabilizing member;

a first parallelogram assembly having a first stabilizing member, a first resilient member and the central stabilizing member, the first parallelogram assembly being hingeably connected to the first and second base plates;

a second parallelogram assembly having a second stabilizing arm, a second resilient member and the central stabilizing member, the second parallelogram assembly being hingeably connected to the first and second base plates, the first and second parallelogram assemblies being disposed offset from each other, the connection points being eccentrically and orthogonally disposed relative to each other, the resilient members being hingeably connected to the connection points, the resilient members being sufficiently resilient to permit movement of the base plates relative to each other so as to counterbalance the arm when a payload is applied to either base plate.

According to another aspect there is provided a positioning apparatus, the apparatus comprising:

at least two actuatable spaced apart counterbalanced arms mounted on a plate in parallel with respect to each other, the counterbalanced arms being in communication with each other;

each arm including two modular base links, each modular base link having:

a first base plate;

a second base plate;

first and second connection points;

third and fourth connection points;

at least two stabilizing members connected to and extending between the first and second base plates; and

at least two non-magnetic resilient members, the first non-magnetic resilient member being hingeably connected to and in communication with the first and second connection points, the second non-magnetic resilient member being hingeably connected to and in communication with the third and fourth connection points, the connection points being eccentrically and orthogonally disposed relative to each other, the resilient members being sufficiently resilient to permit movement of the base plates relative to each other so as to counterbalance the arms when the counterbalanced arms are actuated.

### BRIEF DESCRIPTION OF THE FIGURES

In order that the herein described may be readily understood, embodiments are illustrated by way of example in the accompanying Figures.

FIG. 1 is a perspective view of a counterbalancing arm showing two base links with a payload;

FIG. 2 is a side view of the arm of FIG. 1;

FIG. 3 is a perspective view of a modular base link;

FIG. 4 is an alternative perspective view of the modular base link of FIG. 3;

FIG. 5 is a cross sectional view of the base link;

FIG. 5A is a detailed side view of the orientation of two counterbalancing springs;

FIGS. 6A, 6B and 6C are side views of the base link showing pitch range of motion;

FIGS. 7A, 7B and 7C are top views of the base link showing yaw ranges of motion;

FIG. 8 is a perspective view of a belt driven modular base link;

FIG. 9 is a top perspective view of an alternative belt driven modular base link;

FIG. 10 is a side view of a plurality of alternative belt driven modular base links in a counterbalance arm;

FIG. 11 is a perspective view of the counterbalance arm of FIG. 10 showing base links folded back on themselves in a 360 degree helix;

FIG. 12 is a side view of an alternative counterbalancing arm design;

FIG. 12A is an enlarged detailed view of several links taken from FIG. 9;

FIG. 12B is a cross sectional view taken along line X-X′ in FIG. 9A;

FIGS. 13A, 13B and 13C are respectively a side view of an alternative end link design; a rear view of the link design; and a perspective view of the link design;

FIG. 14 is a perspective view of a double parallelogram linkage;

FIGS. 14A-D illustrates the double parallelogram linkage moving through 360 degrees;

FIG. 15 is a perspective view of a system using two counterbalanced arms in parallel; and

FIGS. 16A and 16B are respectively a rear view of the two counterbalanced arms in parallel; and a side view of the two counterbalanced arms in parallel.

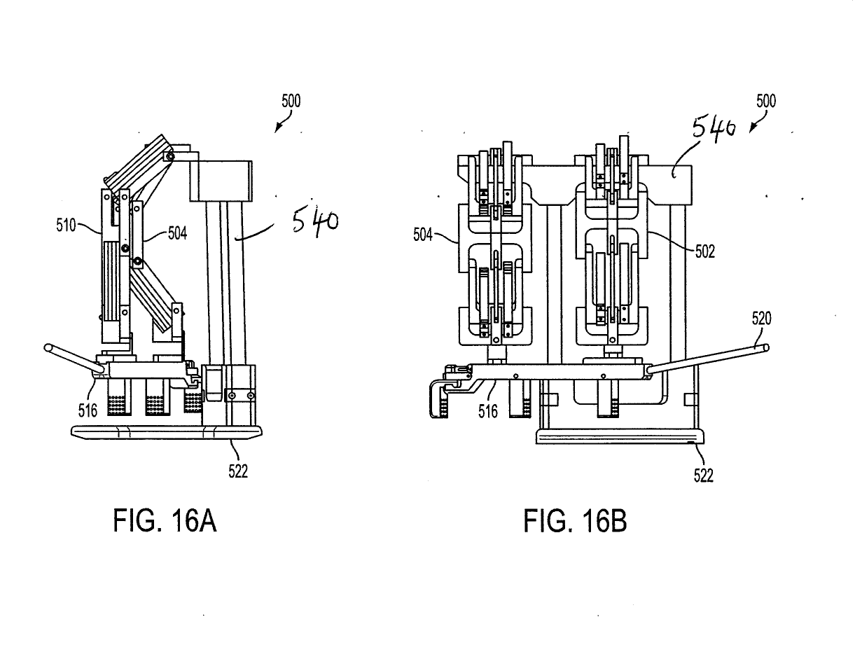

Further details of the modular base link and its advantages will be apparent from the detailed description included below.

### DETAILED DESCRIPTION

Our design can be applied in the design of a fully automated robotic arm for medical applications in which motors can be mounted onto the device to adjust the arm pose. Traditional designs use high torque motors to counterbalance the arm and payload weight creating potential harm for a patient. In the event of a malfunction, these motors may potentially drive the arm into the patient with a minimum force of twice the weight of the arm. In the event of a power failure, a traditional arm may lose its pose and slump under its own weight as the motors can no longer counterbalance the weight. Brakes can be applied to prevent a traditional arm from slumping in a power failure. However, the traditional arm will become fully locked and its pose un-adjustable until power restored. In comparison, our new arm design is passively counterbalanced using springs. As a result, safer low torque motors can be used to drive the new arm design and motors are not required to maintain the robot pose. Furthermore, the new arm can be fully back-drivable allowing the robot pose to be manually adjusted in the event of power failure. Our arm is unique amongst medical robotics since the arm provides an additional intrinsic level of safety over traditional medical robotic designs.

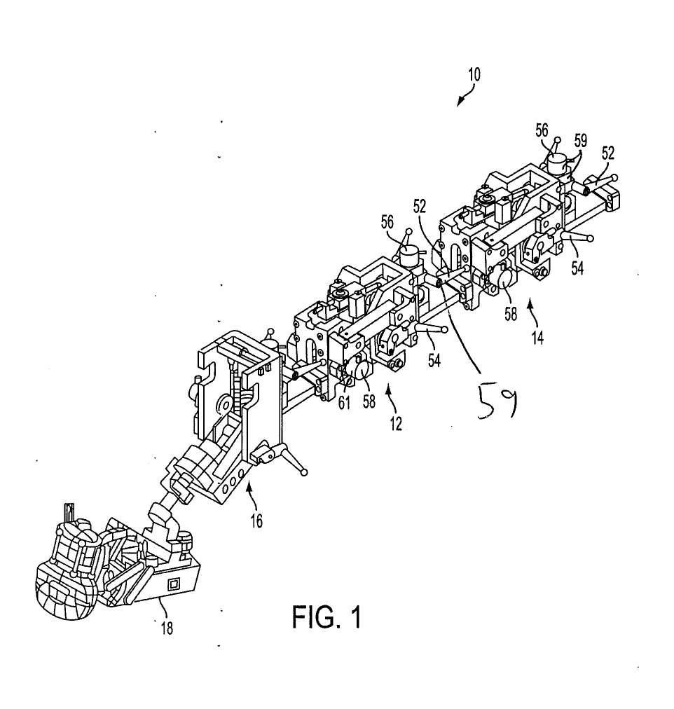

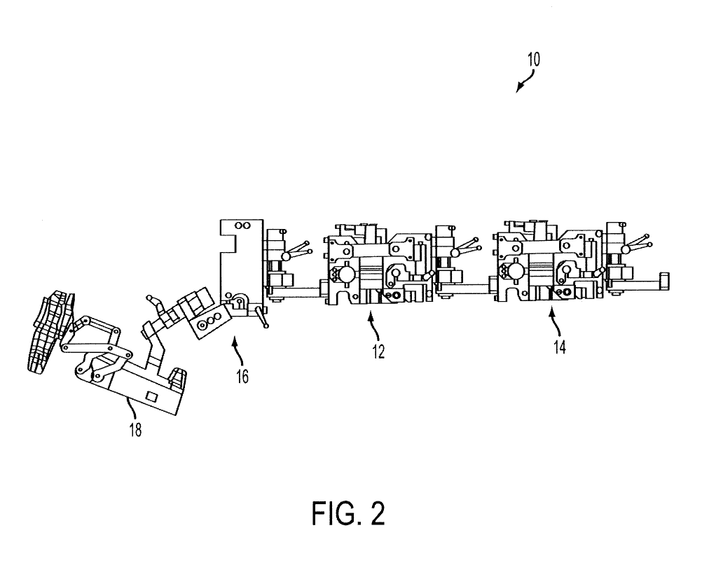

Referring to FIGS. 1 and 2, there is illustrated a counterbalancing arm 10 with two modular base links 12, 14 and an end link 16 connected to the base link 12. Although only two base links 12, 14 are illustrated, it should be pointed out that any number of base links can be used depending on the desired application. The end link 16 is illustrated connected to a payload 18, which in this case is an ultra sound probe and mover. A tool (not shown) may also be located at the end link 16. The end link 16 is located remote from an operator. The end link 16 does not necessarily contain a parallelogram structure. The structure of the end link 16 can be adjusted depending upon the requirements of the application and the payload. The end link 16 allows three rotational degrees of freedom which is required for ultrasound applications. The end link 16 can be modified to allow different motions depending upon the application requirements.

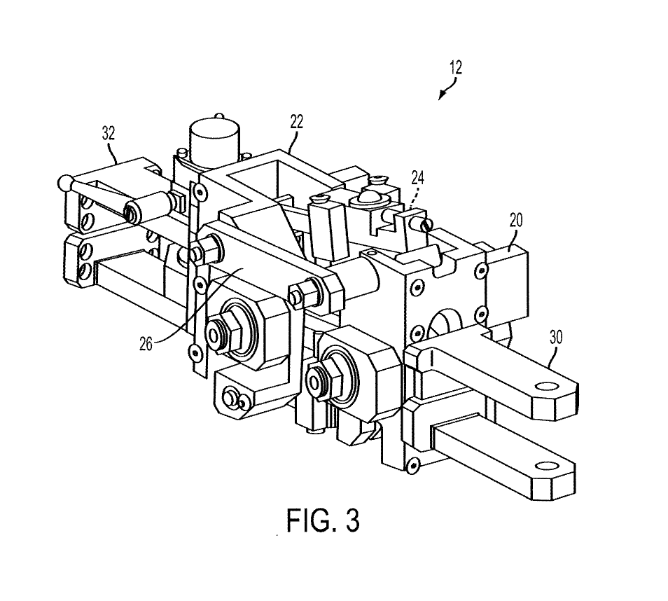

Referring to FIGS. 3, 4, 5 and 5A the base link 12 includes a first base plate 20, a second base plate 22, a first stabilizing member (arm) 24 and a second stabilizing member (arm) 26 which are connected together to form a parallelogram structure 48 and serve to stabilize the base link 12. The first stabilizing arm 24 can be located interior of the base link 12, and the second stabilizing arm 26 can be located exterior of the base link 12. Alternatively, the first stabilizing arm 24 can be located exterior of the base link 12, and the second stabilizing arm 26 can be located interior of the base link 12. Two connector end portions 30, 32 are connected to and extend away from the first base plate 20 and the second base plate 22 respectively. The connector end portions 30, 32 connect adjacent base links together in series. During operation, the first and second base plates 20, 22 remain parallel to each other and the two arms 24, 26 remain parallel to one another. Although not illustrated, an ultrasound transducer can be mounted onto the arm 10. A sonographer could manually adjust the position of the transducer until the desired imaging plane is acquired. The sonographer would then release the transducer and the arm 10 would maintain the transducer position. The use of the arm 10 with a single point of adjustment would be beneficial. It allows adjustment of the spring counterbalance force to apply any necessary downward transducer pressure into the patient. The use of the arm 10 also provides a solution related to prolonged arm abduction, prolonged twisting and application of transducer pressure.

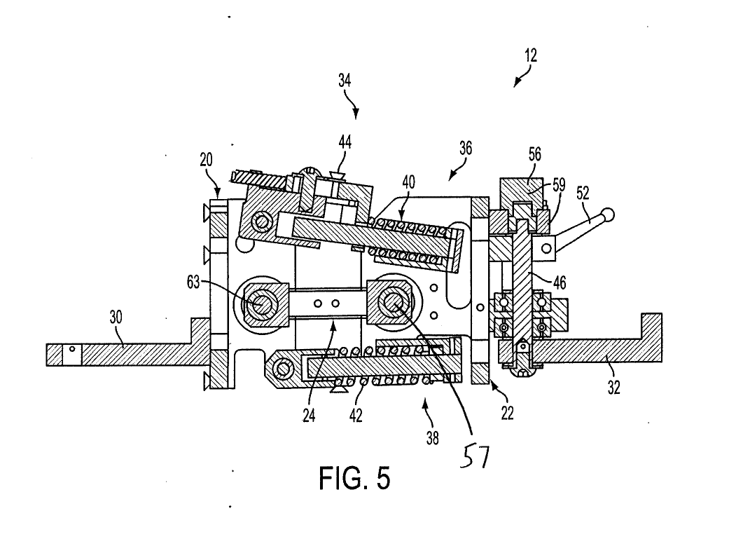

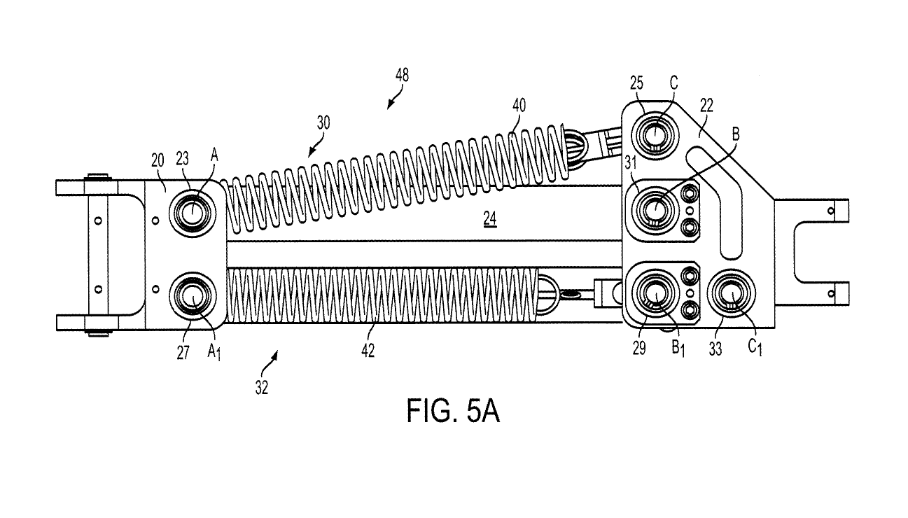

Referring now to FIGS. 5 and 5A, each base link 12, 14 includes a counterbalance assembly 34 which contains first and second resilient counterbalance members 36, 38. As the counterbalance assembly 34 is identical for each base link, only one will be described in detail. In the example illustrated, the resilient counterbalance members 36, 38 are a first counterbalancing spring 40 and a second counterbalancing spring 42. Examples of counterbalance springs include compression springs, extension springs, leaf springs or gas springs. The first counterbalancing spring 40 can be preloaded using a spring adjust 44, which is connected thereto. A counterbalanced payload of each base link can be adjusted by changing the preload of the first counterbalance spring 40. The second counterbalance spring 42 does not require adjustment for preload. Each base link is adjusted independently. The counterbalancing springs 40, 42 are sufficiently resilient to permit movement of the first connector end 30 relative to the second connector end 32. In one example illustrated, the counterbalancing springs 40, 42 are arranged such that the first counterbalancing spring 40 is located above the second counterbalancing spring 42. It is also contemplated that the counterbalancing springs can be located parallel to each other (side-by-side), or indeed any arrangement sufficient to achieve the desired result. Specifically, the first counterbalancing spring 40 is angled away from the second counterbalance spring 42 and are in communication with the respective first and second connector ends 30, 32 to permit movement of the first connector end 30 relative to the second connector end 32 The spring adjust 44 is connected to the first counterbalancing spring 40.

Referring specifically to FIG. 5A, the first and second resilient members 30, 32 are eccentrically hingeably connected to the base plates 20, 22 at connection points A, A1, C and B1. The eccentricities of the resilient members 30, 32 are spaced apart by 90 degrees. The first and second springs 40, 42 are attached to the parallelogram (A, A1, B, B1) such that angle A, C, B is approximately 90 degrees greater than angle A1, B1, C1. In a non-parallelogram linkage, the moment the springs must counteract is equal to the payload mass multiplied by the distance of the payload centre of mass from bearings 29 and 31 in the second base plate 22. For the parallelogram, the total moment the counterbalance springs must support is the length of the stabilizing arm 24 multiplied by the payload mass. The payload can be located anywhere in space and only needs to be anchored to the first base plate 20. Since the stabilizing arm length 24 is shorter than the payload centre of mass distance, the moment the counterbalance spring must counteract is reduced. The counterbalanced arm 12, which includes a number of short parallelogram links is capable of supporting the same payload as an arm containing a single parallelogram link of equal length. Thus, the arm 12 having multiple shorter lengths and shorter counterbalancing springs can also be positioned in a larger workspace with more degrees of freedom than a single long link. This means that an arm with multiple segments will have a greater load carrying capability than a single segment arm or an arm with fewer segments of equal length that uses the same springs as in the single (or fewer) segment arm. The major advantage of the parallelogram arrangement is that counterbalance can be achieved with the location of the payload center of mass at any point.

Theoretically, four hingeable connections are required to form the parallelogram structure 48. In addition, each counterbalance spring 40, 42, must be connected to two spring hinge points A, C and A₁, C1. One of the spring hinge points A, A₁ must be attached to the first base plate 20 and one of the spring hinge points C, C₁ must be attached to the second base plate 22. A hinge point can be shared by both the parallelogram structure 48 and one of the springs in order to reduce the complexity and size of the counterbalance arm 12. A spring can share one or both of its hinge points in common with the parallelogram structure 48. However, the springs 40, 42 could also be optionally mounted such that they share no hinge point in common with the parallelogram structure 48. If no common pivot is used, one of the spring pivots must positioned on a line connecting two parallelogram hinge points. It should be pointed out that the pivot can be positioned anywhere on the two base links. One skilled in the art will recognize that there are many different combinations that the counterbalance springs can achieve, which ultimately provide the same effect.

Although there are an infinite number of different spring arrangements, FIGS. 5 and 5A illustrate only one arrangement Only spring 40 (with the vertical eccentric) can be arranged between the opposite corners of the parallelogram. Spring 42 will require a hinge point independent of the parallelogram hinge points. Because of the nature of the eccentrics, only one spring at a time can be arranged at opposite corners. In this modified configuration, the distance between the hinge connection points A and A1 (or B and B1) replaces the eccentric hinge point C-B, as best illustrated in FIG. 5, thereby producing a more compact linkage. Using many parallelogram containing base links in place of a single arm permits miniaturization of the arm structure while maintaining the arm payload and range capabilities. Short parallelogram arm segments allow smaller springs to successfully counterbalance the payload. The addition of each base link increases the degrees of freedom of the arm, which in turn increases the arm's flexibility and working volume. Advantageously, by applying the counterbalance springs to the parallelogram structure, we have now unexpectedly achieved a number of advantages over the prior art designs noted above. Specifically, Bax et al in United States published patent application number US20100319164A1 merely shows a 90 degree eccentricity, but not on a parallelogram structure.

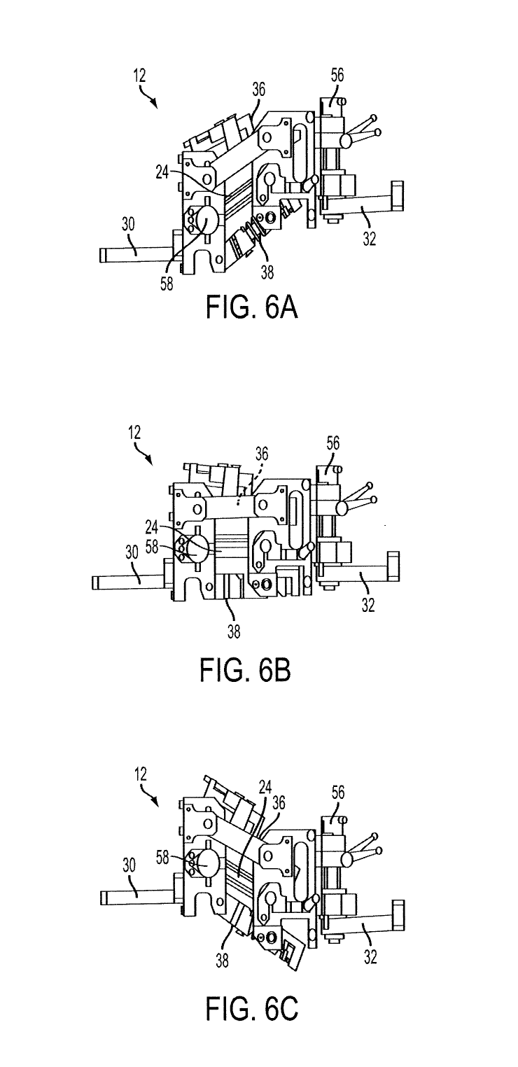

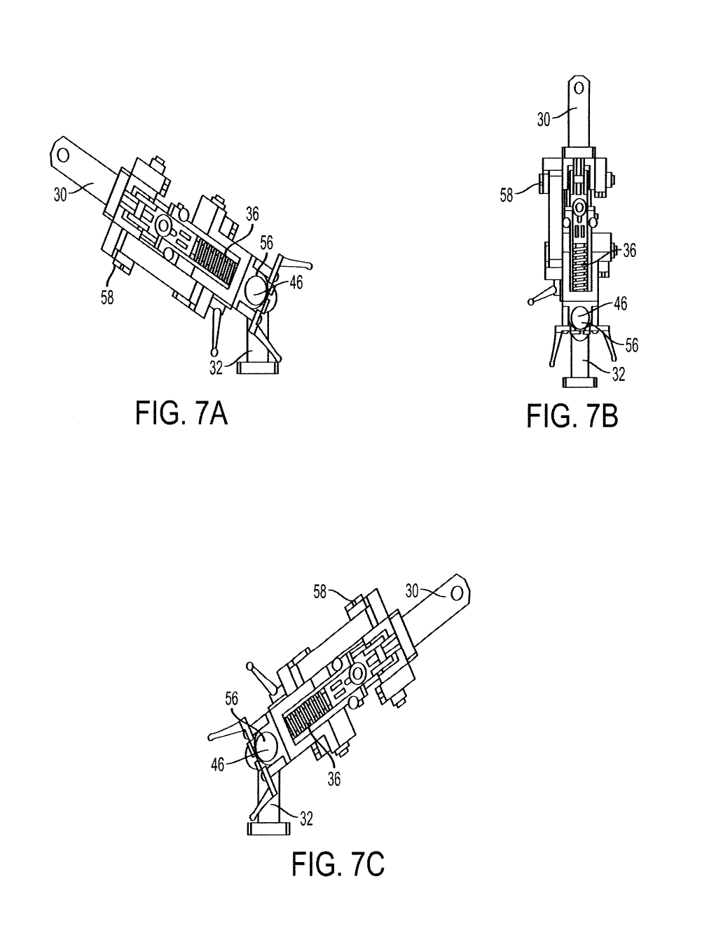

As best illustrated in FIGS. 6A-6C and 7A-7C, the resilient members 36, 38 are sufficiently resilient to permit movement of the first connector end 30 relative to the second connector end 32 in a plurality of pitching motions as illustrated in FIGS. 6A through 6C. A hinged connector 46 is connected to the second base plate 22 and permits hingeable movement (yawing) motion as illustrated in FIGS. 7A through 7C of the base link, which is independent of the pitching movements.

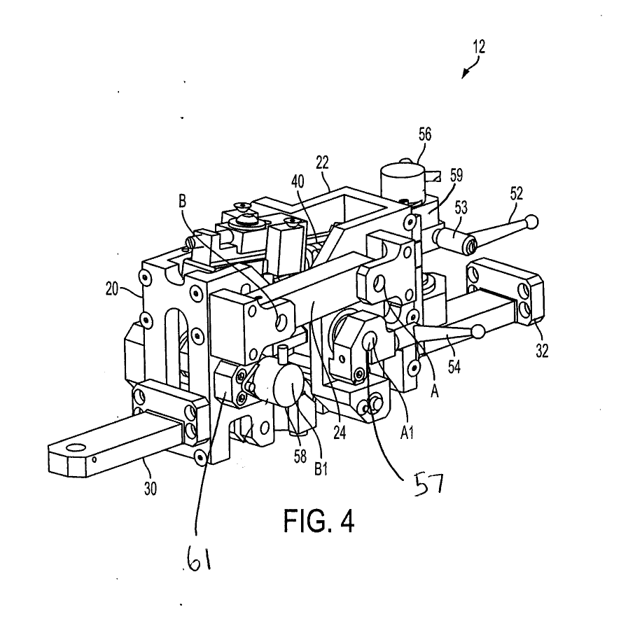

Referring again to FIGS. 4 and 5, the base link 12 can be braked individual or simultaneously. A brake 52 is attached to the hinged connector 46. Another brake 54 acts on the stabilizing arms 24, 26. The brake 54 only directly locks the stabilizing arm 24. To lock the pitch motion, a brake only needs to be attached to one of the stabilizing arms 24 or 26, not both. Finally, the brake 54 directly acts on a shaft 57 which is pinned to the arm 24. The brakes act to rigidify the stabilizing arm and lock the parallelogram. Braking may be manual or automated.

Referring to FIGS. 1 and 4, the arm 10 can be operated as a fully passive, semi-automated or fully automated device. For a passive operation, the arm 10 is adjusted manually and is only used to counterbalance the weight of a tool or payload. For a semi-automated operation, the arm 10 is again manually adjusted by the user while encoders are used to track the orientation of the arm 10 and the payload positions. Two encoders 56, 58 are each mounted on encoder blocks 59, 61 which are connected to the base plate 22,20. The encoder 56 measures the rotation of the hinged connector 46 and the encoder 58 measures the rotation of a large shaft 63 at the centre of the base plate 20. Attached to the end of the hinged connector 46 and to the large shaft 63 are magnets. In one example, the encoders 56,58 measure the rotation of the magnet onto the end of the shafts. Different types of encoders can be used, for example, but not limited to, optical encoders.

Referring to FIGS. 4, 6, and 7, the encoder 58 is attached to the end plate 20 and measures the rotation of the shaft 63 from which the up/down motion can be calculated. The encoder 56 is attached to the plate 22 and measures the rotation of the base link as shown in FIG. 7. The encoders 56 and 58 ensure that the arm 10 remains within a desired workspace or the payload is positioned at a particular target. For a fully automated operation, motors can be used. In one example, the encoders 56, 58 could be a motor/encoder combination; an encoder; or only a motor. In the example with a motor/encoder combination, they would be connected in series. The motors can be used to adjust the arm while the encoders track the arm and payload positions. Each additional base link in the arm 10 increases the arm degrees of freedom by two. The brakes 52, 54 can be applied to each degree of freedom individually. Braking can be accomplished manually, as in the prototype, or automatically, such as using hydraulics. Each degree of freedom is tracked using the encoders 56, 58.

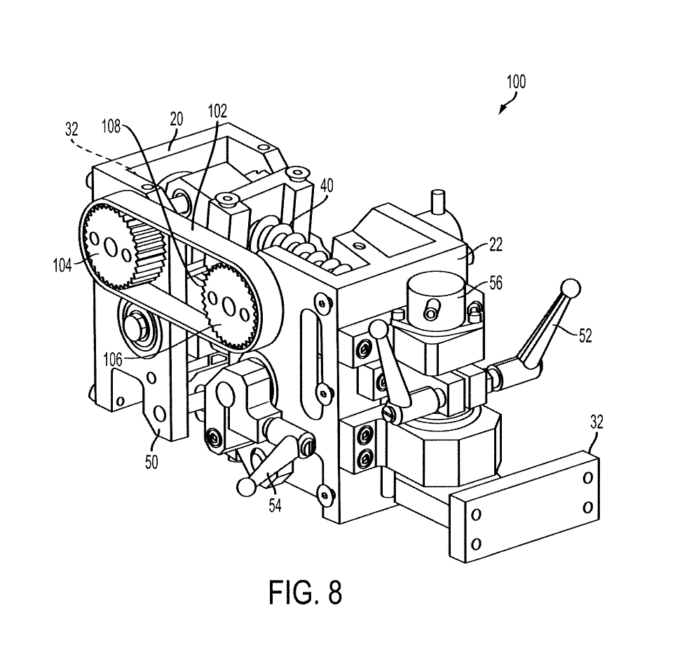

Referring now to FIG. 8, an alternative design of a base link is shown generally at 100. The base link 100 is different from the base link 12 described above in that it includes an endless belt loop 102 located around first and second drive wheels 104, 106 and is moveable to define the parallelogram arrangement. In this example, an internal parallel arm 108 is located between the belt loop 102 to stabilize the base link 100. The drive wheels 104, 106 and the belt loop 102 are located exterior or interior of the base link 100. The belt loop 102 could also be located interior of the base link 100, but this would limit the range of movement.

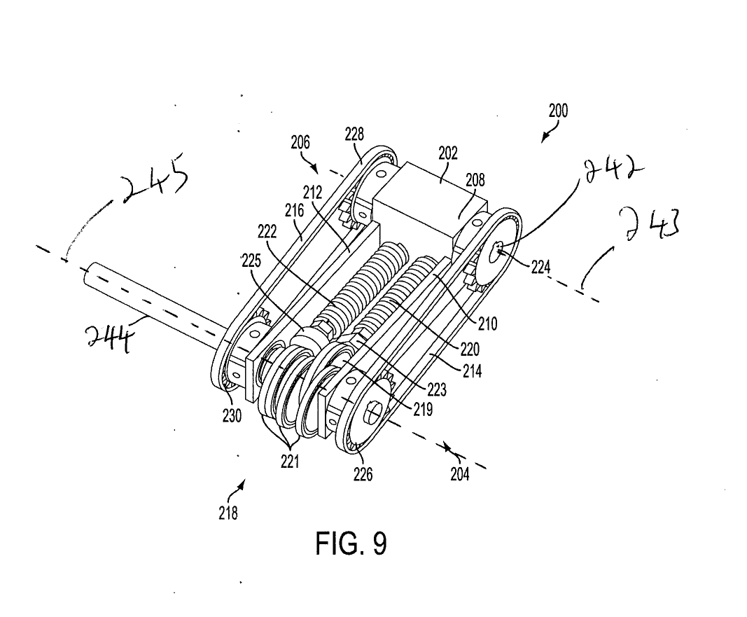

Referring now to FIGS. 9, 10 and 11, which illustrate another alternative base link 200. The base link 200 not only permits movement of each base link throughout 360 degrees, but also permits maintenance of the pose of the payload through 360 degrees. The base link 200 is mounted on a support and includes a body 202 and two wheel assemblies 204, 206. The wheel assemblies 204, 206 are each fixably connected to opposite sides of the body 202. The body 202 is block 208 which includes two side bars 210, 212 located on either side of the block 208, are spaced apart, are parallel to each other and extend away from the block 208.

The first wheel assembly 204 includes first and second spaced apart wheels 224, 226, and a first endless loop 214 mounted on the first and second wheels 224, 226. The second wheel assembly 216 includes third and fourth spaced apart wheels 228, 230 and a second endless loop 216 mounted for rotation about the third and fourth wheels 238, 230. An eccentric cam assembly 218 is fixed relative to the wheels 230, 226 and connected to the wheel assemblies 204, 206. The eccentric cam assembly 218 includes eccentric cams 219, 221 which abut cam followers 223, 225 located at each end of the springs 220, 222. Two resilient members (counterbalance springs) 220, 222 extend between the body 202 and the cam assembly 218. The springs 220, 222 are disposed substantially parallel to each other. One end of each spring 220, 222 is fixably connected to the body 202, while the other end of each spring 220, 222 is abuttingly connected to the cam member 218. The body 202 is located for orbital rotation about the fixed first and fourth wheels 224, 230 and the cam assembly 218, and a first axle 244 having an axis 245. As the body 202 orbitally rotates the springs 220, 222 via the cam followers 223,225 travel along the cams 219, 221 causing the springs 220,222 to compress or relax. Since the wheels form a parallelogram, they do not rotate relative to one another and are a fixed orientation relative to each other.

Still referring to FIGS. 9, 10 and 11, a third wheel assembly 232 connects another base link 201 to the base link 200 The third wheel assembly 232 includes fifth and sixth spaced apart wheels 234, 236. A third endless loop 238 is mounted on the fifth and sixth wheels 234, 236. The third wheel assembly 232 is connected to the first wheel assembly 204 of the base link 200. In the examples illustrated, a plurality of base links are rotably connected to each other. Thus, the third wheel assembly 232 is connected to a body 240 of the adjacent base link 201. A second axle 242 having an axis 243 connects the third wheel assembly 232 to the first wheel assembly 204 of the base link 200. The first wheel 224 and the third wheel 228 and the cam assembly 218 are fixably counted on the first axle 244. The first axle 244 is disposed orthogonal with respect to the springs 220,222. The first connector axle 244 extends away from the fourth wheel 230 and includes a payload end 246 for holding the payload.

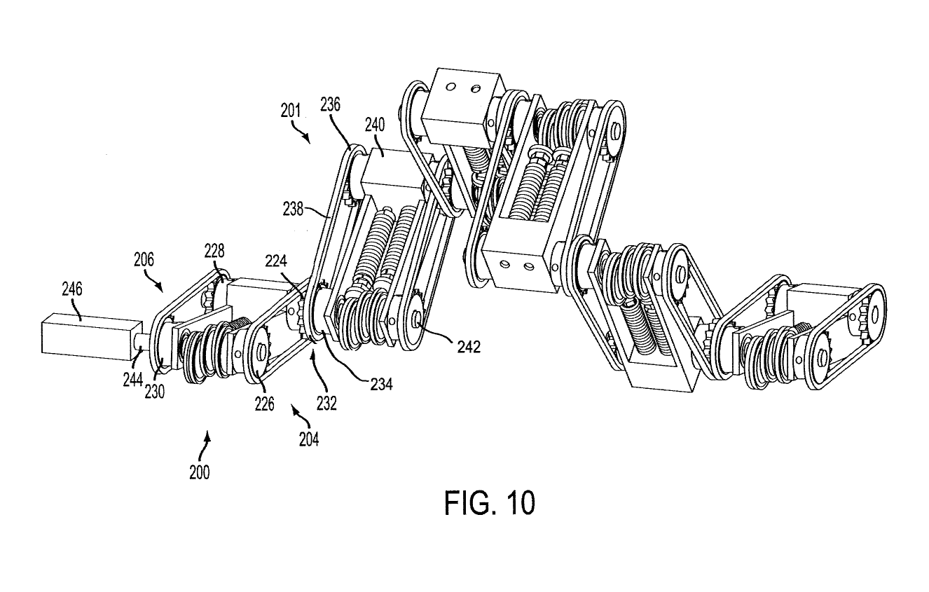

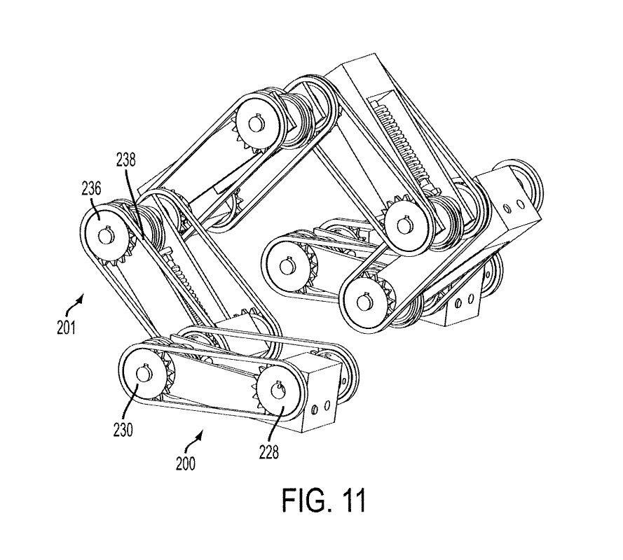

As best seen in FIGS. 10 and 11, a plurality of base links are sequentially connected to each other and are rotatable about respective axles. The base links are orbitally rotatable about 360 degrees. As best illustrated in FIG. 11, the base links can be folded back on themselves in a 360 degree helix.

The endless belt loops 214, 216, 238 and the wheels permit formation of a parallelogram arrangement of the base links. The base links in this design are approximately 6×4×2 inches and have a carrying capacity of approximately 32 pounds, 8 ounces.

Each of the base link designs functions in the same manner where either the stabilizing arms 24, 26 or endless belt loops 102, 214, 216, 238 maintains the relative orientations the first base plate 20 and the second base plate 22.

Generally speaking, the base link designs provide a simpler and more effective solution to counterbalancing of the arm 10 and payload than in conventional designs. The first counterbalancing spring 40 provides an ideal counterbalance of the parallelogram at the horizontal. As the arm 10 moves away from the horizontal, the second counterbalancing spring 42 corrects the first spring 40 for errors in the counterbalancing. The first spring can be adjusted to counterbalance varying payloads and the second spring 42 requires no adjustment. The ability of the second spring 42 to correct for the first spring 40 is consistent throughout the full range of motion of the parallelogram. As a result, our base link designs have no inherent restrictions in the angular range of motion it can support. Moreover, our design is capable of operating over a greater range of motion than conventional designs and without degradation of performance at extreme angles.

Our design can also maintain greater isoelasticity than conventional designs and does not sacrifice isoelasticity to achieve a greater range of motion. Greater isoelasticity improves device usability as the user will be required to apply a consistent force to move the device throughout its full range of motion. Due to improved correction of counterbalancing errors, the arm 10 is far less prone to drifting and will be better able to maintain a pose when released by the user. The ability of the arm 10 to reliably maintain a pose is especially important for applications, such as medicine, where consistency and high accuracy is demanded. The springs can be adjusted for varying payloads either manually or through motorization. This idea is independent of the concept of motorizing the arm joints to adjust orientation. Since the arm is fully counterbalanced, motorization of the joints can be done in a safer manner since lower torque motors are needed to move the joints.

The spring balance contained within each base link can be adjusted to counterbalance varying payloads. Adjustment of the counterbalancing springs can be accomplished using two different approaches. First, the system can be designed so that each counterbalance spring requires independent adjustment by the user. Alternatively, a single point of adjustment can incorporated into the design to allow the user to simultaneously adjust all of the counterbalancing springs within the arm.

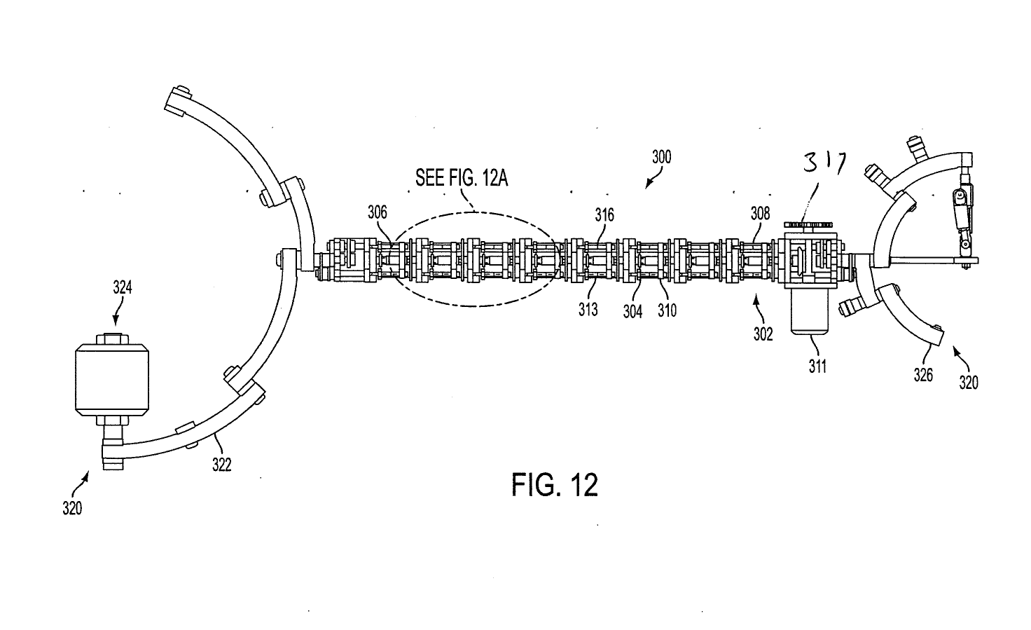

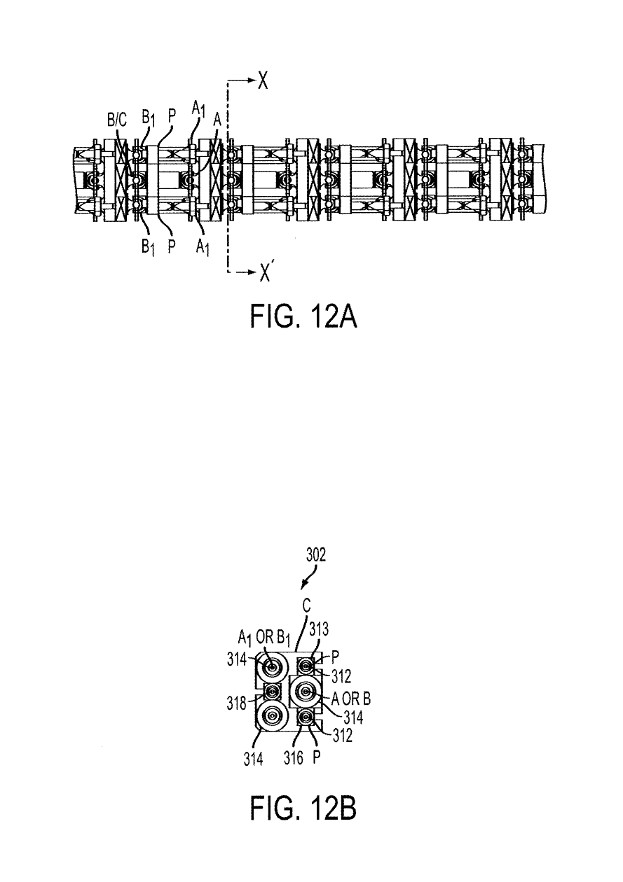

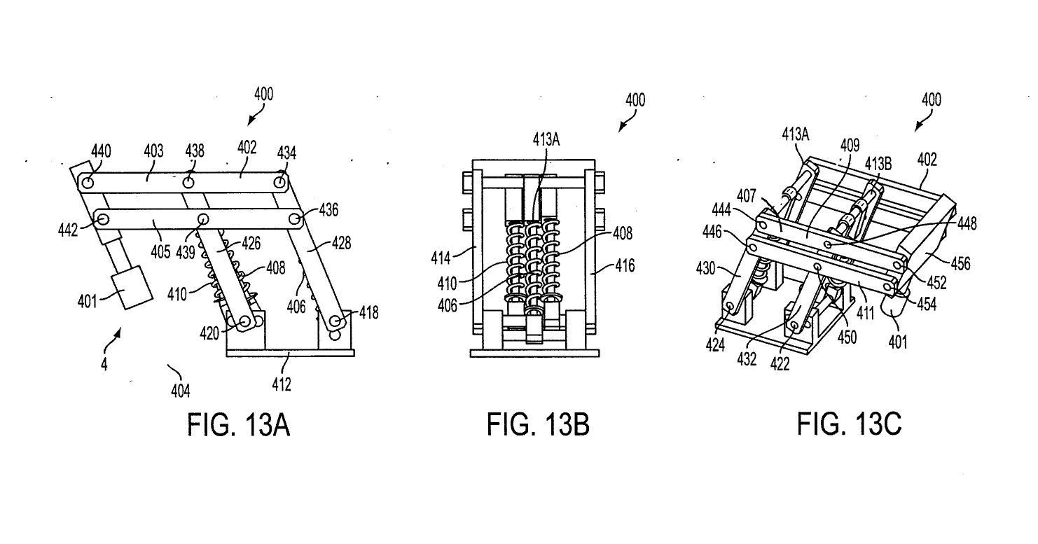

Referring to FIGS. 12, 12A and 12B, an alternative design of the arm is shown generally at 300. In this design, the adjustment of the spring counterbalance mechanism of each link is changed to allow for simultaneous adjustment of all links. Each link 302 contains two independent sources of adjustment for the spring counterbalance. Arm load nuts 304 account for the weight of arm 300 and its constituent components. In addition to the payload, each link 302 in the arm 300 is required to counterbalance the weight of itself and any subsequent links. As a result, the load each link 302 must support arising from the weight of the arm 300 is unique. A final link 308 in the arm 300 supports the entire weight of the arm 300, whereas a first link 306 of the arm 300 only supports the weight of itself. During device calibration, the arm load nuts 304 of each link 302 are manually adjusted using a wrench to account for the weight of the arm 300. Once the arm load nuts 304 are correctly set, they require no further adjustment. Payload nuts 310 account for the weight of payload on the device. The force the spring counterbalance of each link 302 must exert to support the payload is equal. As a result, the amount of adjustment required for the payload nuts 310 of each link is also equal. To avoid unnecessary repetition, the payload nuts 310 can be adjusted from a single common adjustment point. To adjust the payload nuts 310, a base shaft 312 is turned at the base of the arm 300 using either a motor or hand crank 317. The base shaft 312 at the base of the arm is connected to each link through serially connected U-joints 314. The device provides a power lift assist to the operator.

In the designs described above in FIGS. 1 through 5, and 9 through 11, the arm optionally provides an upwards or downwards drift depending on the application. The arm also provides for adjustment to correctly balance the load and experience no drift. However, the previous designs do not incorporate a single point of adjustment. As result, each link in the arm would need to be individually adjusted. However, if the payload and drift requirements are fixed, in the case for example, of an ultrasound probe which must press downwards into the patient, the setup would only be required once. For applications requiring power assist and for changing payload, the design shown in FIG. 12 is used.

For fixed payload and assist requirements, the design shown in FIGS. 1 through 5 above is used.

The arm shown in FIGS. 12, 12A and 12B, allows for frequent single point adjustment which advantageously allows for quick adjustment. The use of power assist is optional; its use depends on the desired application. The payload nuts 310 can be over adjusted causing the arm 300 to tend to drift upwards and ease the lifting of a heavy object. Inversely, the payload nuts 310 can be under adjusted causing drift downwards and ease the lowering of objects. The power lift assist can be user controlled with a switch to cause lift upwards or downwards by driving a motor 311 at the single point of adjustment. The power list assist can also be automated with mechatronic control. Sensors such as strain gauges can detect motion and drive the adjustment motor to cause lift or decline.

Still referring to FIGS. 12 and 12A, the U-joints 314 for payload adjust are located at the center of each link 302. As the base shaft 312 is turned, the payload nuts 310 in each link 302 will be advanced thereby increasing the payload support of each link 302. The base shaft 312 can be turned in the opposite direction to retract the payload nuts 310 and lower the payload support. The payload nuts 310 must be adjusted each time the arm payload is changed. In this arm design, three springs 313, 316, 318 are used in place of two to ensure symmetrically distribution of counterbalancing forces. Mathematically, the two springs 313, 316 that are in parallel on the sides of each link can be considered to be a single spring that exerts twice the force. This minimizes the effects of twisting of the arm 300 due to the spring forces acting on the mechanism and also reduces the friction within the adjustment mechanism that would be caused from the twisting loads of a single spring acting asymmetrically on one side of the linkage.

Still referring to FIG. 12, an alternative end link 320 design for the arm 300 is illustrated. The end link 320 consists of a forward spherical linkage 322 (or gimbal joint) which directly supports the payload. Rotational axes of the spherical linkage 322 rotate about a common point 324 in space called a Remote Center of Motion (RCM). The spherical linkage 322 contains two rotational degrees of freedom to adjust the orientation of the payload. However, the forward spherical linkage 322 requires counterbalancing to prevent it from collapsing under the weight of payload. The simplest method to counterbalance the spherical linkage 322 is to position the center of mass of the payload at the RCM 324. However, if the payload must be positioned offset from the RCM 324, further hardware is required to counterbalance the linkage 322. A rear spherical linkage 326 is connected to the base of the device. The rear spherical linkage 326 is coupled to the forward spherical linkage 322 using two sets of serially connected U-joints 328. Each set of serially connected U-joints 328 transmits the motion of one of the rotational degrees of freedom of the spherical linkage 322. The rear linkage 326 functions as a pantograph and mimics in mirror-image the motions of the forward linkage 322. The payload spring counterbalance provides a force to counterbalance the weight of the payload.

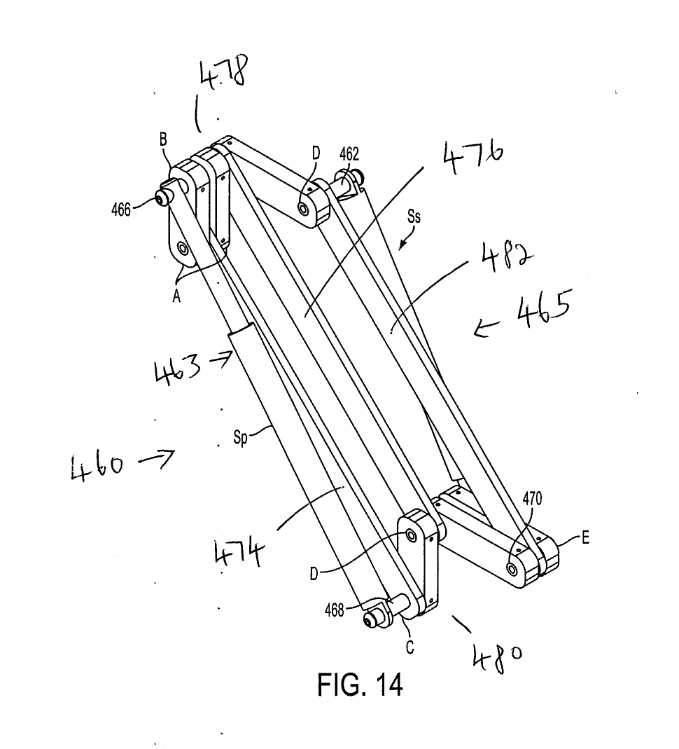

Referring now to FIGS. 13A, 13B and 13C, an alternative end link design 400 which includes a first parallelogram assembly 402 having first and second substantially parallel arms 403,405. It should be pointed out that although this design illustrates two parallelogram structures, more than two can also be used. The first parallelogram assembly 402 is hingeably connected to a base 412. A second parallelogram assembly 407 includes third and fourth substantially parallel arms 409, 411. It should be pointed out that although two arms are illustrated, more than two arms are also possible and that the number of parallel arms in the first parallelogram assembly does not need to equal the number of arms in the second parallelogram assembly. The second parallelogram assembly 407 is hingeably connected to the base 412. The second parallelogram assembly 407 is spaced apart from, and parallel to, the first parallelogram assembly 402 and is connected to it using spacer bars 413A, 413B, which extend between the two assemblies 402, 407. Each end of the spacer bars 413A, 413B is connected to the respective first and second parallelogram assemblies 402,407. Two resilient members (counterbalance springs) 408,410 are located in substantial parallelism and extend between the base 412 and the spacer 413B. A third spring 406 extends between the base 412 and the spacer bar 413A. Each spring 408, 410 has two ends which are pivotally connected to the base 412 and to the respective spacer bar, 413B. The parallelogram assemblies 402, 407 form a RCM 404, which is illustrated for manipulating a payload 401. Although this design can be used as an end link on the arm 200, it could also be used in a separate independent device. Furthermore, this design can also be used on the end of the arm 10. The parallelogram assemblies 402, 407 are counterbalanced using the same spring counterbalance concept used in the base links described above. The end link design 400 for the arm 200 is used in place of the spherical linkage, and the parallelogram assemblies 402,407 forming the RCM 404 can be used. The parallelogram assemblies 402,407 are a very common design amongst medical robotics for creating the RCM 404. An advantage of the parallelogram end link 400 is that it can be counterbalanced using a much simpler design compared to the spherical linkage described above. The parallelogram assemblies 402,407 do not require the rear mirror-image linkage or the serially connected U-joints to transmit counterbalancing forces. Although in FIGS. 13A-C, the four arms 403,405,409,411 are illustrated, it should be noted that two arms are equivalent. In this case, three springs 406, 408, 410 allow the arm 200 and the payload 401 to be counterbalanced on either side of the vertical. With two springs, the arm and payload are only counterbalanced on one side of the vertical.

Still referring to FIGS. 13A. 13B and 13C, the base 412 has four connection points 418,420,422,424 The first and second arms 403,405 have six connection points 434,436,438,439,440,442. The second arm 409,411 has six connection points 444, 446, 448, 450, 452, 454. A first support arm 430 is movably connected to the first and second arms 403, 405 at the two connection points. 444,446 A second support arm 432 is movably connected to the first and second arms 403, 405 at the two other connection points 448,450. The second support arm 432 is located away from the first support arm 430. The first and second arms 403,405 and the first and second support arms 430,432 define a first parallelogram. The third and fourth support arms 426,428 are identical to the first and second arms and will not be described in detail. The two spacer bars 413A, 413B are connected to the first, second, third and fourth arms at the connection points. Two additional spacer bars are connected to the first, second, third and fourth arms at the other connection points. The spacer bars space the arms apart in substantial parallelism. The springs 408,410 are connected to two base connection points and to the second spacer bar 413B. A payload end 456 is connected to the ends of the arms at the four connection points 440,442,452,454 the arms and the support arms are movable about their respective connection points to move the payload 401 in a desired direction.

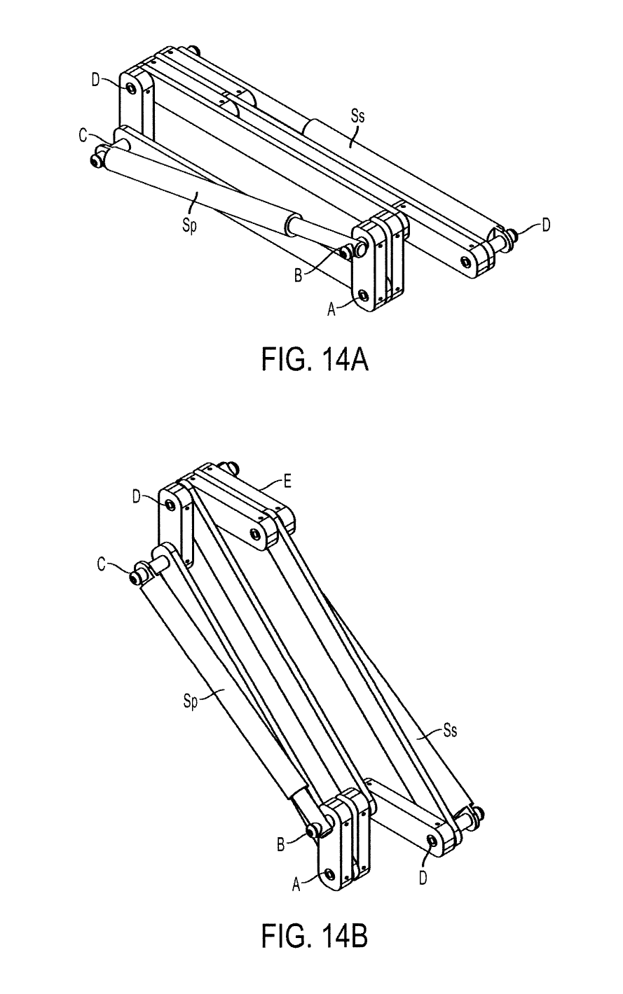

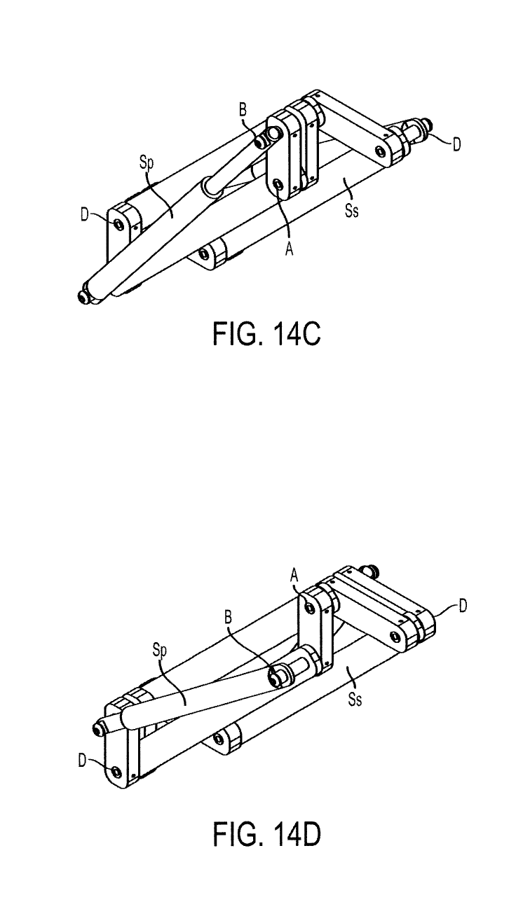

Referring now to FIGS. 14, 14A through 14D, a counterbalancing arm that includes a double parallelogram linkage 460 is illustrated. The linkage 460 permits movement through 360 degrees. The arm includes two parallelogram assemblies 463, 465. The first parallelogram assembly 463 includes a first stabilizing arm 474 and a first resilient member Sp and a central stabilizing member 476. In one example, the first parallelogram assembly 463 is hingeably connected to first and second base plates 478,480. The second parallelogram assembly 465 includes a second stabilizing arm 482, a second resilient member Ss and the central stabilizing member 476, which is located between the first and second stabilizing members 474,482. The stabilizing arms and the resilient members are disposed substantially parallel to each other. The second parallelogram assembly 465 is hingeably connected to the first and second base plates 478, 480. The first and second parallelogram assemblies 463, 465 are disposed offset from each other by at least 20 degrees. The first and second base plates 472, 474 with two spaced apart connection points each, i.e., A, B, C, D and B, D, E, F, connected to each other such that A B D and C D E are pinned through each shaft 462,466,468,470 to form two rigid links with three holes and are arranged such that angle ABD (and CDE) is 90 degrees. This connection creates two parallelograms that are 90 degrees out of phase to each other. This angle does not have to be 90 degrees but is typically greater than zero and less than 180 degrees. Typically, 90 degree is used because the springs can attach directly to the parallelogram hinge points. The connection points are eccentrically and orthogonally disposed relative to each other. As with the designs described above, the resilient members Sp and Ss are sufficiently resilient to permit movement of the base plates relative to each other so as to counterbalance the arm when a payload is applied to either base plate. In one example, resilient members Sp and Ss are compression springs which are each adapted to be used in compression or extension modes. The stabilizing arms 474,476, 482 are stabilizing arms. The first stabilizing arm 463 and the first resilient member Sp are hingeably connected to the central stabilizing member 476. The second stabilizing member 482 and the second resilient member Ss are also hingeably connected to the central stabilizing member 476 In one example, the stabilizing members and the resilient members are all connected to common connection points on the base plates. In another example, the stabilizing members and the resilient members are all connected to different connection points on the base plates.

As best illustrated in FIG. 14, the first parallelogram assembly 460 includes three first parallelogram assembly base plates 484, 486, 488 each having two spaced apart connection points A, B. The first stabilizing member 474, the first resilient member Sp and the central stabilizing member 476 are hingeably connected to the three first parallelogram assembly base plates 484, 486, 488. The second parallelogram assembly 465 includes three second parallelogram assembly base plates 490,492,494 each having two spaced apart connection points D,E. The second stabilizing member 482, the second resilient member Ss and the central stabilizing member 476 hingeably connected to the second parallelogram assembly base plates 490,492,494.

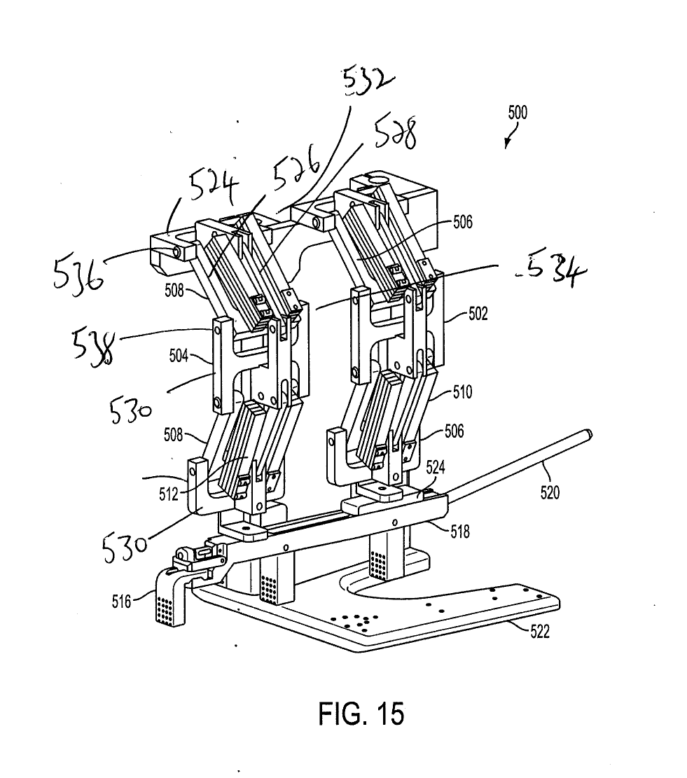

Referring to now to FIGS. 15, 15A and 15B, a positioning apparatus 500 using two counterbalanced arms 502, 504 operating in parallel is illustrated. This apparatus also uses the parallelogram structure as described above. Each arm 502, 504 contains two base links 506, 508 and instead of metal compression springs, non-metallic plastic leaf springs 510, 512 are used in the spring counterbalance. The plastic leaf springs 510, 512 permit the apparatus 500 to operate within the bore of a Magnetic Resonance (MR) scanner or any other magnetically sensitive environment. Plastic leaf springs can be used in this design to achieve MR capability. However, metallic leaf springs can be used for non-magnetically sensitive applications. The apparatus 500 has been developed to complete MRI-guided focal therapy of the prostate. Needle templates 516 (a payload end) hold and secure a needle used for focal therapy. The arm 504 is used to adjust the position of the needle tip insertion point on the skin surface of the patient. The arm 502 is used to adjust the trajectory of the needle for insertion into the patient. The arm 504 is pinned to a template holder 518 whereas the arm 502 is pinned to a sliding dovetail rail 524 to increase the device workspace. Although not shown, brakes can be installed to independently fix the arms once they are in the desired position. The apparatus 500 is manually manipulated by the physician using a handle 520. However, both arms 502, 504 can be motorized and encoded to fully automate the needle positioning. The compactness of the arm would allow it to operate within the limited space available within an MRI bore. A base 522 provides stability to the device 500.

The modular base links described herein can be applied in the design of a fully automated robotic arm for medical and non-medical applications. Motors can be mounted onto the device to adjust the arm pose. Because the arm design is passively counterbalanced using springs, safer low torque motors can be used to drive the new arm design and motors are not require dot maintain the robot pose. Furthermore, the arm can be fully back-drivable allowing the robot pose to be manually adjusted in the event of power failure. The arm provides an additional intrinsic level of safety over traditional medical robotic designs. Thus, the apparatus includes the at least two actuatable spaced apart counterbalanced arms 502,504 mounted on the base plate 522 in parallel with respect to each other. The counterbalanced arms 502,504 in communication with each other, each including the two modular base links 506,508. Each of the modular base links 506,508 includes a first base plate 524, a second base plate 530; first and second connection points 532, 534; third and fourth connection points 536,538. At least two stabilizing arms 526, 528 are connected to and extend between the first and second base plates 524, 530. The first non-magnetic spring 510 is hingeably connected to and in communication with the first and second connection points 532, 534. The second non-magnetic spring 512 is also hingeably connected to and in communication with the third and fourth connection points 536,538. As with the previously described designs, the connection points are eccentrically and orthogonally disposed relative to each other. The leaf springs are sufficiently resilient to permit movement of the base plates 524,530 relative to each other so as to counterbalance the arms when the counterbalanced arms are actuated using the handle 520. As with the previously described designs, the stabilizing arms 526,528 are hingeably connected to the base plates 524,530 so as to define two parallel parallelogram structures in each counterbalancing arm. A support 540 connects the two counterbalancing arms together and is located rearwardly of the counterbalancing arms. Although two arms are illustrated, it is to be understood that a plurality of actuatable spaced apart counterbalanced arms can also be used, depending on the application.

Although the above description relates to a specific preferred embodiment as presently contemplated by the inventor, it will be understood that the invention in its broad aspect includes mechanical and functional equivalents of the elements described herein.

What is claimed is:

1. A modular base link for use in a counterbalancing arm, the base link comprising:
a first base plate;
a second base plate;
first and second connection points;
third and fourth connection points;
at least two stabilizing members connected to and extending between the first and second base plates; and
at least two resilient members, the first resilient member being hingeably connected to and in communication with the first and second connection points, the second resilient member being hingeably connected to and in communication with the third and fourth connection points, the connection points being eccentrically and orthogonally disposed relative to each other, the resilient members being sufficiently resilient to permit movement of the base plates relative to each other so as to counterbalance the arm when a payload is applied to either base plate.

2. The base link, according to claim 1, in which first and second connector ends are connected to the respective first and second base plates.

3. The base link, according to claim 1, in which the stabilizing members are stabilizing arms hingeably connected to the base plates and extend therebetween.

4. The base link, according to claim 2, in which the first connector end is connected to another base link and the second connector end is connected to an end link.

5. The base link, according to claim 2, in which the counterbalancing arm includes a plurality of serially connected base links.

6. The base link, according to claim 3, in which the stabilizing arms are hingeably connected to the base plates so as to define the base link as parallelogram structure.

7. The base link, according to claim 3, in which the stabilizing arms are disposed parallel to each other.

8. The base link, according to claim 1, in which the first and second resilient members are eccentrically connected to the base plates and are spaced apart by 90 degrees.

9. The base link, according to claim 1, in which the first and second resilient members are connected between the two stabilizing members.

10. The base link, according to claim 1, in which the first and second resilient members are connected to either of the first or second base plates and one of the stabilizing members.

11. The base link, according to claim 1, in which the first resilient member is located above the second resilient member.

12. The base link, according to claim 1, in which the resilient members are disposed side-by-side to each other.

13. The base link, according to claim 6, in which the resilient members are disposed outside of the parallelogram structure.

14. The base link, according to claim 6, in which the resilient members are located inside the parallelogram structure.

15. The base link, according to claim 1, in which the first and second resilient members are counterbalancing springs.

16. The base link, according to claim 15, in which the counterbalancing springs are compression springs, extension springs, leaf springs, or gas springs.

17. The base link, according to claim 1, in which the first resilient member is adjustable using a spring adjust to counterbalance varying loads.

18. The base link, according to claim 1, in which the second resilient member corrects errors in counterbalancing of the first resilient member.

19. The base links, according to claim 1, in which first and second handbrakes are connected to either the first base plate or the second base plate to permit locking of the base link in a desired orientation.

20. The base link, according to claim 1, in which two encoders are connected to the base link to track the orientation of the arm and the position of the payload.

21. The base link, according to claim 2, in which the resilient members are sufficiently resilient to permit movement of the first connector end relative to the second connector end in a plurality of pitching motions.

22. The base link, according to claim 2, includes a hinged connector connected to the first base plate.

23. The base link, according to claim 22, in which the hinged connector permits hingeable movement of the base link in a plurality of yawing motions.

24. The base link, according to claim 1, includes at least one wheel assembly.

25. The base link, according to claim 24, in which the wheel assembly includes first and second spaced apart wheels connected to the first and second base plates; and an endless belt loop located around the first and second drive wheels so as to form a parallelogram structure.

26. The base link, according to claim 25, in which the endless loop is located exterior of the base link.

27. The base link, according to claim 25, in which the first endless loop is located interior of the base link.

28. The base link, according to claim 1, includes at least one load adjustment member.

29. The base link, according to claim 28, in which the load adjustment member includes at least one arm load adjustment nut to account for the weight of the arm, and at least one payload adjustment nut to account for the weight of the payload.

30. The base link, according to claim 29, in which the load adjustment member further includes a rotatable shaft connected to the payload adjustment nuts.

31. The base link, according to claim 30, in which the rotatable shaft is located at the base of the arm and is connected to each link in a plurality of base links through serially connected U-joints.

32. The base link, according to claim 31, in which the U-joints are located to permit hingeable movement of each base link so that as the shaft is rotated in a first direction, the payload adjustment nuts in each link are advanced to increase the payload support of each base link.

33. The base link, according to claim 30, in which the rotatable shaft is rotated in a second direction opposite the first direction to retract the payload adjustment nuts and lower the payload support.

34. The base link, according claim 30, in which two springs are mounted on respective rotatable shafts to permit symmetrical distribution of counterbalancing forces, a third spring is located between the first and the second base plates.

35. The base link, according to claim 34, in which the two springs are disposed parallel to each other.

36. The base link, according to claim 30, In which the adjustment nuts are serially connected.

37. The base link, according to claim 4 in which the end link includes a forward spherical linkage for supporting the payload, the forward spherical linkage having spherical axes which are rotatable about a common point in space

38. The base link, according to claim 37, further includes a rear spherical linkage connected to a base, the rear spherical linkage being connected to the forward spherical linkage using two sets of serially connected U-joints, each set of serially connected U-joints are 90 degrees out of phase to permit the transmission of rotational motion from base link to base link between the rear spherical linkage and the forward spherical linkage.

39. The base link, according to claim 1, in which the hingeable connections are formed using at least two U-joints.

40. A modular base link for use in a counterbalancing arm, the base link comprising:
a body;
at least one wheel assembly having first and second spaced apart wheels and a first endless loop mounted on the first and second wheels, the first wheel assembly being fixably mounted on a support, the first wheel assembly being in communication with the body;
an eccentric cam assembly connected to the first wheel, the eccentric cam assembly having a plurality of eccentric cams that are orthogonally disposed relative to each other; and
at least two resilient members extending between the body and the eccentric cam assembly, one end of each resilient member being connected to the body, the other end of each resilient member being abuttingly connected to the eccentric cam assembly,
the body being located for orbital rotation about the first wheel and the eccentric cam assembly so as to cause the resilient members to travel along the eccentric cam assembly.

41. The base link, according to claim 40, in which the cams are disposed 90 degree apart.

42. The base link, according to claim 40 includes two wheel assemblies, the first wheel assembly includes the first and second wheels and a first endless loop; and third and fourth wheel and a second endless loop mounted on the third and fourth wheels.

43. The base link, according to claim 42, in which:
the first wheel and the third wheels, and the cam assembly are fixably mounted on a first axle, the first axle being disposed orthogonal to the resilient members.

44. The base link, according to claim 43, in which the first axle includes a payload end for holding the payload

45. The base link, according to claim 42, in which the second wheel and the fourth wheel are fixably mounted on a second axle.

46. The base link, according to claim 45, in which the second axle connects another base link thereto.

47. The base link, according to claim 46, includes a plurality of base links sequentially connected to each other and orbitally rotatable about respective connector axles.

48. The base link, according to claim 47, in which the base links are orbitally rotatable about 360 degrees.

49. The base link, according to claim 47, in which the endless belt loops and the wheels permit formation of a parallelogram structure of the base links.

50. A link comprising:
a base;
at least two parallel support arms hingeably connected to the base;
a first parallelogram assembly having at least two substantially parallel arms hingeably connected to the support arms at four arm connection points; and
at least two resilient members located in substantial parallelism, one resilient member being hingeably connected to and in communication with first and second resilient member connection points, the other resilient member being hingeably connected to and in communication with third and fourth resilient member connection points, the connection points being eccentrically and orthogonally disposed relative to each other, the resilient members being sufficiently resilient to permit movement of the first and second support arms relative to each other.

51. The link, according to claim 50, further includes:
at least two support arms hingeably connected to the base; and
a second parallelogram assembly having at least two substantially parallel arms, the second parallelogram assembly being hingeably connected to the third and fourth support arms at four arm connection points, the second parallelogram assembly being disposed parallel to the first parallelogram assembly.

52. The link, according to claim 51, in which:
at least one spacer extends between the first and second parallelogram assemblies, each end of the spacer being connected to the respective first and second parallelogram assemblies.

53. The link, according to claim 51, in which the base has four connection points.

54. The link, according to claim 50, in which:
a payload end is hingeably connected to the ends of the parallel arms at two payload connection points, the parallel arms and the support arms being hingeably movably about their respective connection points to move the payload in a desired direction.

55. The link, according to claim 51, in which:
a payload end is hingeably connected to the ends of the parallel arms at four payload connection points, the parallel arms and the support arms being hingeably movably about their respective connection points to move the payload in a desired direction.

56. The link, according to claim 50, in which the link includes a parallelogram linkage to permit formation of a remote center of motion.

57. The link, according to claim 51, in which the link includes a parallelogram linkage to permit formation of a remote center of motion.

58. The link, according to claim 50, further includes a third resilient member.

59. The link, according to claim 50, in which the link is an end link located at the end of a counterbalancing arm.

60. A counterbalancing arm for payload positioning, the arm comprising:
at least two modular base links connected together for movement relative to each other, each base link having: i) two base plates; ii) four connection points; iii) at least two stabilizing members connected to and extending between the base plates; and iv) at least two resilient members, the first resilient member being hingeably connected to and in communication with two of the connection points, the second resilient member being hingeably connected to and in communication with the other two connection points, the connection points being eccentrically and orthogonally disposed relative to each other, the resilient members being sufficiently resilient to permit movement of the base plates relative to each other so as to counterbalance the arm when a payload is applied to either base plate.

61. The arm, according to claim 60, includes a plurality of modular base links.

62. The arm, according to claim 60, in which the resilient members are counterbalancing springs.

63. A counterbalancing arm comprising:
first and second base plates, each base plate having two spaced apart connection points;
a central stabilizing member;
a first parallelogram assembly having a first stabilizing member, a first resilient member and the central stabilizing member, the first parallelogram assembly being hingeably connected to the first and second base plates;
a second parallelogram assembly having a second stabilizing arm, a second resilient member and the central stabilizing member, the second parallelogram assembly being hingeably connected to the first and second base plates, the first and second parallelogram assemblies being disposed offset from each other, the connection points being eccentrically and orthogonally disposed relative to each other, the resilient members being hingeably connected to the connection points, the resilient members being sufficiently resilient to permit movement of the base plates relative to each other so as to counterbalance the arm when a payload is applied to either base plate.

64. The counterbalancing arm, according to claim 63, in which:
the first stabilizing member and the first resilient member are hingeably connected to the central stabilizing member; and
the second stabilizing member and the second resilient member are hingeably connected to the central stabilizing member.

65. The counterbalancing arm, according to claim 63, in which the stabilizing members and the resilient members are connected to common connection points on the base plates.

66. The counterbalancing arm, according to claim 63, in which the stabilizing members and the resilient members are connected to different connection points on the base plates.

67. The counterbalancing arm, according to claim 63, in which the stabilizing members are disposed parallel to each other.

68. The counterbalancing arm, according to claim 63, in which the central stabilizing member is disposed between the first and second stabilizing members.

69. The counterbalancing arm, according to claim 63, in which the first parallelogram assembly includes three first parallelogram assembly base plates each having two spaced apart connection points, the first stabilizing member, the first resilient member and the central stabilizing member being hingeably connected to the three first parallelogram assembly base plates.

70. The counterbalancing arm, according to claim 63, in which the second parallelogram assembly includes three second parallelogram assembly base plates each having two spaced apart connection points, the second stabilizing member, the second resilient member and the central stabilizing member being hingeably connected to the second parallelogram assembly base plates.

71. The counterbalancing arm, according to claim 63, in which the first and second resilient members are compression springs adapted form compression or extension.

72. The counterbalancing arm, according to claim 63, in which the stabilizing members are stabilizing arms.

73. A positioning apparatus, the apparatus comprising:
at least two actuatable spaced apart counterbalanced arms mounted on a plate in parallel with respect to each other, the counterbalanced arms being in communication with each other;
each arm including two modular base links, each modular base link having:
a first base plate;
a second base plate;
first and second connection points;
third and fourth connection points;
at least two stabilizing members connected to and extending between the first and second base plates; and
at least two non-magnetic resilient members, the first non-magnetic resilient member being hingeably connected to and in communication with the first and second connection points, the second non-magnetic resilient member being hingeably connected to and in communication with the third and fourth connection points, the connection points being eccentrically and orthogonally disposed relative to each other, the resilient members being sufficiently resilient to permit movement of the base plates relative to each other so as to counterbalance the arms when the counterbalanced arms are actuated.

74. The apparatus, according to claim 73, in which the two non-magnetic resilient members are plastic leaf springs.

75. The apparatus, according to claim 73, in which the stabilizing members are stabilizing arms hingeably connected to the base plates so as to define two parallel parallelogram structures in each counterbalancing arm.

76. The apparatus, according to claim 73, is slidably mounted on a rail.

77. The apparatus, according to claim 76, in which a handle is connected at one end of the rail; a payload end is connected to the other end of the rail, the handle being located to permit movement of the counterbalancing arms.

78. The apparatus, according to claim 73, in which the two counterbalancing arms are connected to a support

79. The apparatus, according to claim 73, in which is for MRI-guided focal therapy of the prostate gland.

80. The apparatus, according to claim 73, includes a plurality of actuatable spaced apart counterbalanced arms.
